(19) United States
(12) Patent Application Publication
(10) Pub. No.: US 2006/0079805 A1
(43) Pub. Date: Apr. 13, 2006

(54) Site marker visable under multiple modalities

(76) Inventors: Michael E. Miller, Trafalgar, IN (US); Michael Hoffa, Brownsburg, IN (US); Joseph L. Mark, Indianapolis, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 10/964,087

(22) Filed: Oct. 13, 2004

(51) Int. Cl. A61B 10/00; A61B 6/00

(52) U.S. Cl. 600/562; 600/431

(57) ABSTRACT

An intracorporeal site marker that is adapted to be implanted into a biopsy cavity includes a plurality of balls or particles. The balls or particles are either sintered together or bonded together. Other alternative embodiments of site markers visible under various imaging modes are also disclosed.

### FIELD OF THE INVENTION

The present invention relates generally to site markers for breast biopsy procedures. More specifically, the present invention relates to site markers that are visible under multiple modalities.

### BACKGROUND OF THE INVENTION

In the diagnosis and treatment of breast cancer, it is often necessary to perform a biopsy to remove tissue samples from a suspicious mass. The suspicious mass is typically discovered during a preliminary examination involving visual examination, palpation, X-ray, magnetic resonance imaging (MRI), ultrasound imaging or other detection means.

When a suspicious mass is detected, a sample is taken by biopsy, and then tested to determine whether the mass is malignant or benign. This biopsy procedure can be performed by an open surgical technique, or through the use of a specialized biopsy instrument. To minimize surgical intrusion, a small specialized instrument such as a biopsy needle is inserted in the breast while the position of the needle is monitored using fluoroscopy, ultrasonic imaging, X-rays, MRI or other suitable imaging techniques.

In a relatively new procedure, referred to as stereotactic needle biopsy, the patient lies on a special biopsy table with her breast compressed between the plates of a mammography apparatus and two separate X-rays are taken from two different points of reference. A computer then calculates the exact position of the mass or lesion within the breast. The coordinates of the lesion are then programmed into a mechanical stereotactic apparatus which advances the biopsy needle into the lesion with precision. At least five biopsy samples are usually taken from locations around the lesion and one from the center of the lesion.

Regardless of the method or instrument used to perform the biopsy, subsequent examination of the surgical site may be necessary, either in a follow up examination or for treatment of a cancerous lesion. Treatment often includes a mastectomy, lumpectomy, radiation therapy, or chemotherapy procedure that requires the surgeon or radiologist to direct surgical or radiation treatment to the precise location of the lesion. Because this treatment might extend over days or weeks after the biopsy procedure, and the original features of the tissue may have been removed or altered by the biopsy, it is desirable to insert a site marker into the surgical cavity to serve as a landmark for future identification of the location of the lesion.

Known biopsy site markers have been found to have disadvantages in that the site markers are not visible under all available modalities. Moreover, because of this problem, when cancer is found at a biopsy site that has been previously marked with a site marker, due to the poor visibility of the biopsy site marker under ultrasound or other visualization modalities, the patient must undergo an additional procedure that places an additional device the biopsy site to enable the surgeon to find the biopsy site in subsequent procedures. One known technique has been to place a breast leasion localization wire at the biopsy site. The localization wire is typically placed at the biopsy site via mammography and/or ultrasound.

Accordingly, there is a need for site markers made from biocompatible materials that are visible under various modes of imaging to reduce the number of procedures that patients must undergo in detection and treatment of cancer.

### SUMMARY OF THE INVENTION

Intracorporeal site markers are provided for implantation into a surgical biopsy cavity. In accordance with one aspect of the invention, the site markers include a plurality of balls or particles, bonded together to form a unitary body. The balls or particles are made from biocompatible materials such as titanium, stainless steel or platinum and are visible under multiple modes of imaging. The balls or particles are generally bonded together by sintering or by an adhesive material such as epoxy. Because the inventive site marker is constructed of material that is visible under multiple modalities, there is no need for the patient to be subjected to an additional procedure or have an additional device implanted at the biopsy site to enable the surgeon to locate the biopsy site at a later time.

Alternative embodiments include a site marker having at least one continuous strand of wire that is formed in a molding cavity. The wire is made from any biocompatible material such as titanium, stainless steel, platinum, or other suitable material, and is compressed to form a mass that resembles a ball of yarn. Additionally, the site marker can take the form of a resonating capsule, or a rod with drilled holes.

### BRIEF DESCRIPTION OF THE DRAWINGS

These and other features and advantages of the invention will be apparent from the following detailed description and the appended claims, taken in conjunction with the accompanying drawings, in which:

FIG. 1 is a perspective view of a biopsy site in a human breast showing the breast in section and one or more site markers being implanted in the biopsy cavity using a site marker delivery system;

FIG. 2A is a side elevational view of a site marker according to a first embodiment of the present invention;

FIG. 2B is an end elevational view of the site marker of FIG. 2A;

FIG. 3A is a side elevational view of a site marker according to a second embodiment of the present invention;

FIG. 3B is an end elevational view of the site marker of FIG. 3A;

FIG. 4A is a side elevational view of a site marker according to a third embodiment of the present invention;

FIG. 4B is an end elevational view of the site marker of FIG. 4A;

FIG. 5 is a front elevational view of a site marker according to a fourth embodiment of the present invention;

FIG. 6 is a side elevational view of a site marker according to a fifth embodiment of the present invention;

FIG. 6A is a side elevational view of a site marker according to a sixth embodiment of the present invention;

FIG. 7 is a perspective view of a site marker according to a seventh embodiment of the present invention;

FIG. 7A is a perspective view of a site marker according to an eighth embodiment of the present invention;

FIG. 8A is a side elevational view of a site marker according to a ninth embodiment of the present invention;

FIG. 8B is an end elevational view of the site marker of FIG. 8A.

FIG. 9 is a side elevational view of a site marker in accordance with a tenth embodiment of the present invention

### DETAILED DESCRIPTION OF THE INVENTION

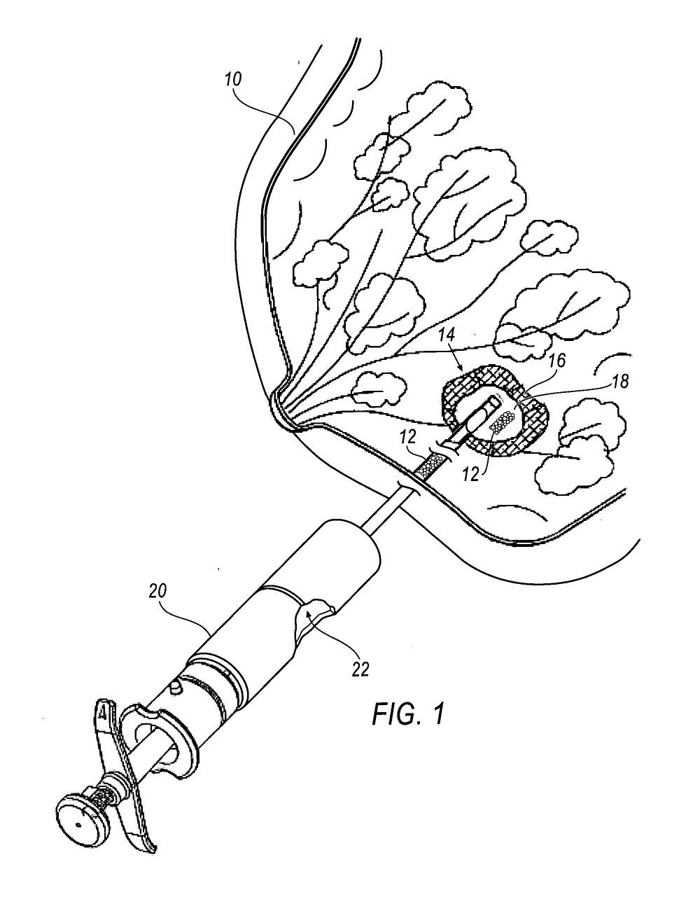

FIG. 1 illustrates a perspective view of a human breast 10 being implanted with a site marker 12 according an embodiment of the present invention. At a biopsy site 14 is a lesion 16 from which a tissue sample has been removed, resulting in a biopsy cavity 18. One or more site markers 12 are implanted in the biopsy cavity 18 using a marker delivery system 20, as shown in FIG. 1. In one embodiment, the marker delivery system 20 is slidably advanced through an inner lumen 22 of a biopsy device (not shown), which avoids the need to withdraw the biopsy device and thereafter insert the marker delivery system 20. Delivering the site marker 12 in the biopsy cavity 18 without withdrawing the biopsy device reduces the amount of tissue damage and enables more accurate placement of the site marker 12. The marker delivery system 20 illustrated in FIG. 1 is exemplary only and it is understood that the site marker embodiments disclosed herein are suitable for use with other marker delivery systems.

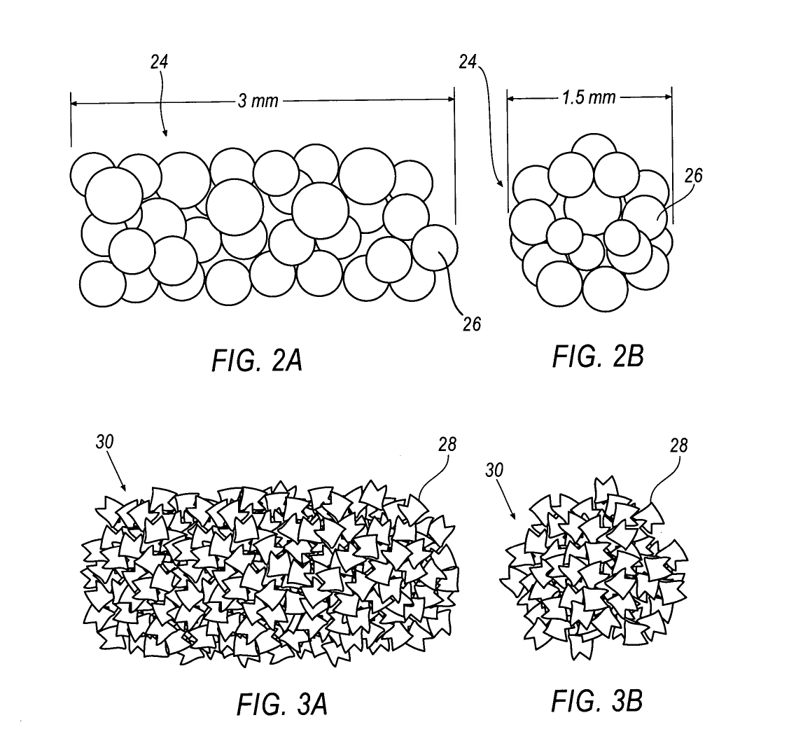

FIGS. 2A-8B illustrate suitable exemplary site marker embodiments according to the present invention. In general, the site markers described herein are made from biocompatible materials such as, but not limited to, titanium, stainless steel, and platinum. These materials have appropriate densities for radiographic imaging, appropriate surface characteristics for ultrasonic imaging, and appropriate magnetic characteristics for magnetic resonance imaging. The site markers that will be described below are preferably made from titanium; however, it is understood that any suitable biocompatible material may be used.

Referring initially to FIGS. 2A and 2B, a site marker 24 includes a plurality of balls 26 sintered together to form a unitary body. The balls 26, as shown, vary in size and are sintered together randomly such that there is no structured or predetermined equidistance between the centers of the balls 26. In other embodiments, the size of the balls 26 may be generally uniform, or the balls 26 may be sintered together such that the centers of the balls 26 are aligned in a predetermined manner. As illustrated in FIGS. 2A and 2B, one embodiment of site marker 24 measures approximately 1.5 mm in diameter (FIG. 2B) and 3 mm in length (FIG. 2A). As those skilled in the art will appreciate, when the size and sintering pattern of the balls 26 are modified, the size, shape and dimensions of the site marker will also vary. The balls 26 may be constructed from any biocompatible material with suitable echogenic properties such as, but not limited to, titanium, stainless steel, or platinum.

FIGS. 3A and 3B illustrate another embodiment of the invention having irregularly shaped particles or bits 28 that are sintered together to form site marker 30. The particles, as shown in FIGS. 3A and 3B, are exaggerated to illustrate the random shapes of the particles 28. In application, however, the edges of the particles are sufficiently smooth so as to not damage any tissue. The particles can be substantially similar in size and shape, or they may vary as shown in FIGS. 3A and 3B. The particles 28 may be constructed from any biocompatible material with suitable echogenic properties such as, but not limited to, titanium, stainless steel, or platinum.

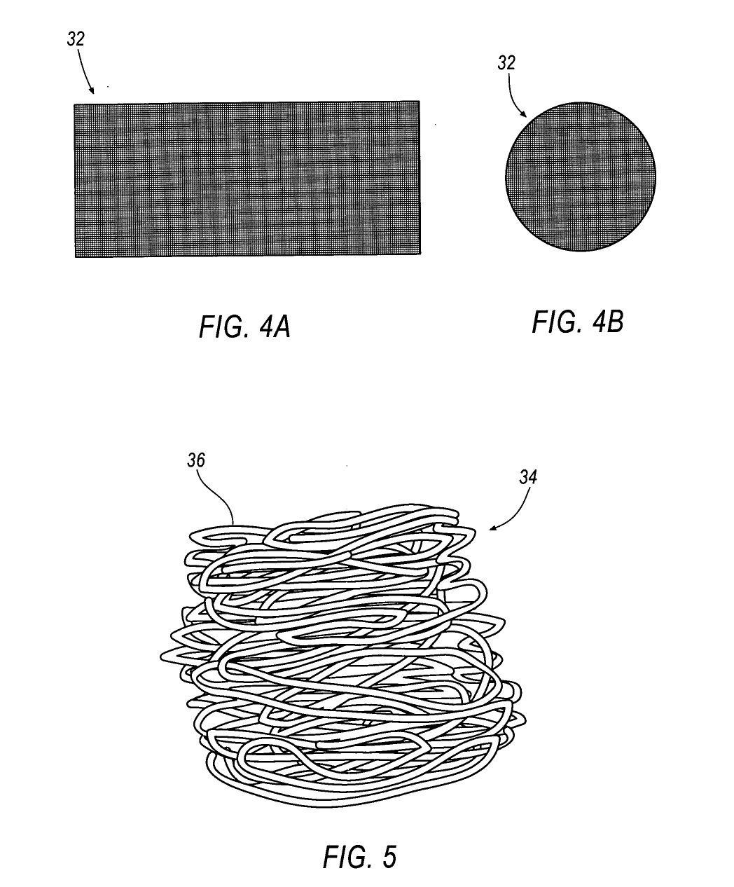

In another aspect of the invention, the particles 28 may be sufficiently small such that, when sintered together, the resultant site marker 32 appears to form a porous metal, as shown in FIGS. 4A and 4B.

FIG. 5 shows another embodiment of a biopsy site marker 34 made from a continuous strand of wire 36. To form the biopsy site marker 34, the wire 36 is fed into a molding cavity (not shown). When the wire 36 reaches the back wall of the cavity, it folds over onto itself conforming to the shape of the molding cavity. The wire 36 is compressed into a mass that resembles a ball of yarn. Inherently, the size and shape of the site marker 34 is dependent upon the size and shape of the molding cavity. The wire 36 may be constructed from any biocompatible material with suitable echogenic properties such as, but not limited to, titanium, stainless steel, or platinum.

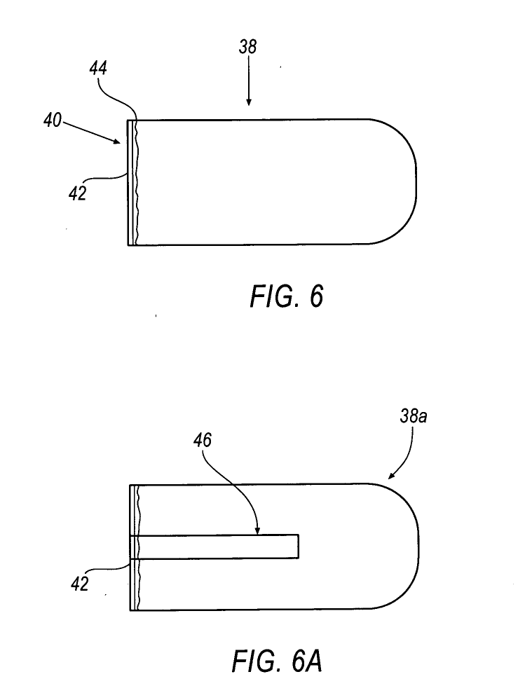

FIG. 6 shows a thin-walled hollow site marker in the form of a capsule 38 having an open end 40. A cap 42 is attached to the open end 40 by a weld 44. The capsule 38 is designed to resonate at a predetermined ultrasound frequency. In the event that the capsule 38 needs to resonate at more than one frequency, a resonant beam 46, as shown in FIG. 6A, can be attached to the inner surface wall of the cap 42 so that the beam resonance is transmitted through the wall of the capsule. The capsule 38 may be constructed from any biocompatible material with suitable echogenic properties such as, but not limited to, titanium, stainless steel, or platinum.

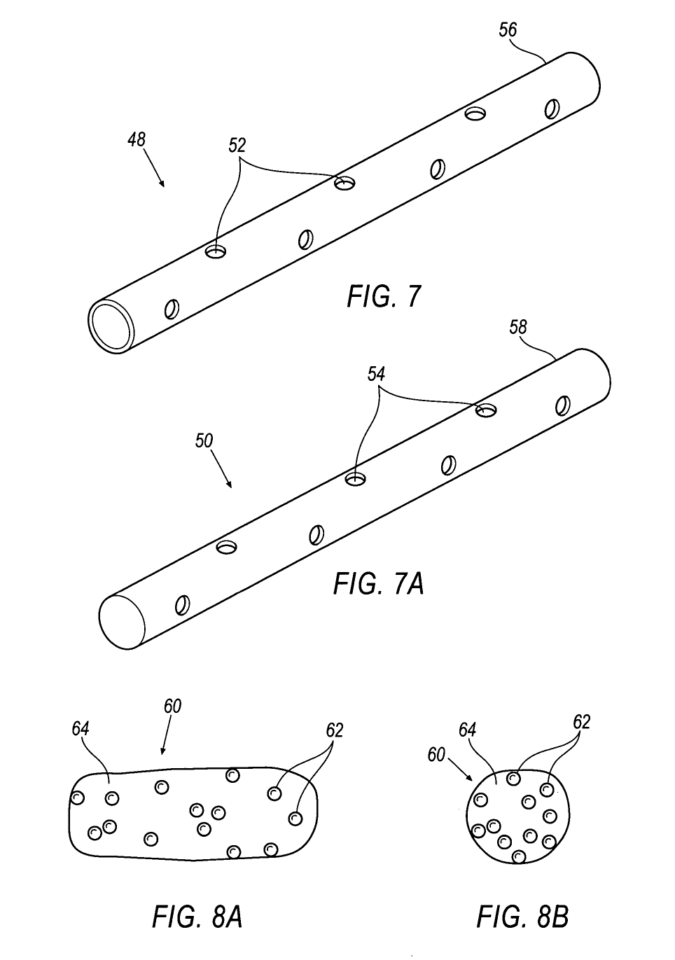

FIGS. 7 and 7A show site marker 48, 50 in the form of a rod 56, 58 having drilled holes 52, 54 throughout the body of the rod. Site marker 48 of FIG. 7 is a solid rod, whereas site marker 50 of FIG. 7A is a hollow rod or tube. The holes in both rods 48, 50 may be drilled in a random or in a predetermined pattern. The rod 56, 58 may be constructed from any biocompatible material with suitable echogenic properties such as, but not limited to, titanium, stainless steel, or platinum.

FIGS. 8A and 8B illustrate another embodiment of a site marker 60 that includes ball or bits 62 of material that are visible under one or more imaging modalities, and dispersed in a block of material 64 that is different than the balls or bits 62. The balls or bits 62 may be constructed of titanium, stainless steel or other suitable material that are visible under more than one imaging modalities. In addition, the balls or bits 62 of material may be contacting each other within the block 64 and may vary in size and shape. In one embodiment, the block of material 64 is a biocompatible material such as epoxy. In another embodiment, the block of material is constructed of a bioabsorbable material that is absorbed by the patient's body such that only the bills 62 remain at the biopsy site.

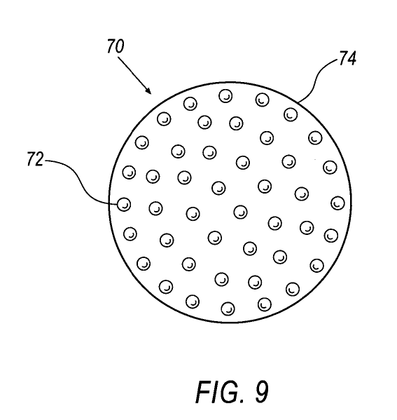

FIG. 9 illustrates another embodiment of a site marker 70 that is made in accordance with the present invention. Site marker 70 is a unitary body made of biocompatible material or a combination of biocompatible materials that are visible under one or more imaging modalities. Maker 70 may be hollow or solid. According to one aspect of the invention, marker 70 further includes a plurality of depressions 72 formed on an outer surface 74 of marker 70. Depressions 72 may be formed on surface 74 so as to be set a predetermined distances apart from one another or may be randomly formed on outer surface 74. Depressions 72 may also be formed so as to have a variety of shapes. In one embodiment, depressions 72 have a parabola shape, with a length of at least about 0.25 mm.

While the present invention has been particularly shown and described with reference to the foregoing preferred embodiments, it should be understood by those skilled in the art that various alternatives to the embodiments of the invention described herein may be employed in practicing the invention without departing from the spirit and scope of the invention as defined in the following claims. It is intended that the following claims define the scope of the invention embodiments within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiment is illustrative, and no single feature or element is essential to all possible combinations that may be claimed in this or a later application.

1. An intracorporeal site marker for implantation in a biopsy cavity, comprising:
a plurality of particles;
wherein said particles are sintered together to form a unitary body.

2. The site marker of claim 1, wherein said particles are sintered together in a random pattern.

3. The site marker of claim 1, wherein said particles are sintered together in a predetermined pattern.

4. The site marker of claim 1, wherein said particles are of uniform size.

5. The site marker of claim 1, wherein at least some of said particles differ in size.

6. The site marker of claim 1, wherein the shape of said particles is varied.

7. The site marker of claim 1, wherein the shape of said particles is generally spherical.

8. The site marker of claim 1, wherein the length of said site marker is approximately twice the diameter thereof.

9. The site marker of claim 8, wherein said site marker measures approximately 1.5 mm in diameter and 3 mm in length.

10. The site marker of claim 1, wherein the material forming said particles is a biocompatible material or a combination of biocompatible materials that are visible under multiple modalities.

11. The site marker of claim 10, wherein the biocompatible material is selected from the group comprising titanium, stainless steel, platinum, and composites and combinations thereof.

12. The site marker of claim 1, wherein said particles are sufficiently small to form an essentially solid and porous marker when sintered together.

13. An intracorporeal site marker for implantation in a biopsy cavity, comprising:
at least one strand of wire that is formed into a predetermined shape so as to form a mass of compressed wire, wherein said strand of wire is not visibly identifiable by an imaging modality as a strand of wire in an uncompressed state.

14. The site marker of claim 13, wherein the material forming said wire is a biocompatible material or a combination of biocompatible materials that are visible under multiple modalities.

15. The site marker of claim 14, wherein the biocompatible material is selected from the group comprising titanium, stainless steel, platinum, and composites and combinations thereof.

16. An intracorporeal site marker implanted into a biopsy cavity, comprising:
a rod having at least one hole therein.

17. The site marker of claim 16, wherein the material forming said rod is a biocompatible material or a combination of biocompatible materials that are visible under multiple modalities.

18. The site marker of claim 17, wherein the biocompatible material is selected from the group comprising titanium, stainless steel, platinum, and composites and combinations thereof.

19. The site marker of claim 16, wherein said holes are arranged in a predetermined pattern.

20. The site marker of claim 16, wherein said rod is a hollow tube.

21. An intracorporeal site marker for implantation into a biopsy cavity, comprising:
a thin wall hollow capsule having an elongated body;
said capsule having a closed first end and an open second end;
a cap welded to close said second open end; and
wherein said capsule is adapted to resonate at a preselected ultrasound frequency.

22. The site marker of claim 18, wherein the material forming said capsule is a biocompatible material or a combination of biocompatible materials that are visible under multiple modalities.

23. The site marker of claim 22, wherein the biocompatible material is selected from the group comprising titanium, stainless steel, platinum, and composites and combinations thereof.

24. The site marker of claim 21, wherein said capsule further includes a resonant beam attached to an inner surface wall of said cap.

25. An intracorporeal site marker for implantation into a biopsy cavity, comprising:
a plurality of particles that are suspended in a second material that is different than the material of said particles to form a unitary body.

26. The site marker of claim 25, wherein said second material is an epoxy.

27. The site marker of claim 25, wherein said second material is a biocompatible material or a combination of biocompatible materials.

28. The site marker of claim 25, wherein said second material is a bio-absorbable material.

29. The site marker of claim 25, wherein the material forming said particles is a biocompatible material or a combination of biocompatible materials that are visible under multiple modalities.

30. The site marker of claim 29, wherein the biocompatible material is selected from the group comprising titanium, stainless steel, platinum, and composites and combinations thereof.

31. The site marker of claim 25, wherein said particles are of uniform size.

32. The site marker of claim 25, wherein at least some of said particles differ in size.

33. The site marker of claim 25, wherein the shape of each of said particles is varied.

34. The site marker of claim 25, wherein the shape of said particles is generally spherical.

35. An intracorporeal site marker for implantation into a biopsy cavity, comprising:
a unitary body that is construction of a biocompatible material that is visible under one or more imaging modalities when said unitary body is positioned within the body;
wherein said unitary body further includes a plurality of depressions formed on an outside surface of said unitary body.

36. The site marker of claim 35, wherein said depressions are formed at predetermined locations on said outside surface of said unitary body.

37. The site marker of claim 35, wherein said depressions have a parabola shape.

38. The site marker of claim 35, wherein the biocompatible material is selected from the group comprising titanium, stainless steel, platinum, and composites and combinations thereof.

39. The site marker of claim 35, wherein said depressions have a length of at least about 0.25 mm.
